(19) United States
(12) Patent Application Publication
(10) Pub. No.: US 2014/0018588 A1
(43) Pub. Date: Jan. 16, 2014

(54) ISOMERIZATION PROCESS FOR HYDROCARBONS WITH RECYCLING OF HYDROGEN HALIDES

(71) Applicant: BASF SE, Ludwigshafen (DE)

(72) Inventors: Katharina Spuhl, Forest (BE); Markus Schmitt, Heidelberg (DE); Jochen Bürkle, Mannheim (DE); Joni Joni, Sulzbach (DE); Steffen Tschrischwitz, Mannheim (DE); Daniel Pfeiffer, Neustadt (DE); Steffen Bitterlich, Dirmstein (DE); Lukas Schulz, Mannheim (DE); Michael Hübner, Lampertheim (DE)

(21) Appl. No.: 13/937,247

(22) Filed: Jul. 9, 2013

Related U.S. Application Data
(60) Provisional application No. 61/670,134, filed on Jul. 11, 2012.

(51) Int. Cl. C07C 5/29

(52) U.S. Cl. CPC C07C 5/29
USPC 585/318

(57) ABSTRACT

The present invention relates to a process for isomerizing at least one hydrocarbon in the presence of an acidic ionic liquid and at least one hydrogen halide (HX) in an apparatus (V1), wherein the hydrogen halide (HX) is removed in an apparatus (V2) in gaseous form from the isomerization product and is at least partly recycled into apparatus (V1).

This patent application claims the benefit of pending U.S. provisional patent application Ser. No. 61/670,134 filed on 11 Jul. 2012, incorporated in its entirety herein by reference.

The present invention relates to a process for isomerizing at least one hydrocarbon in the presence of an acidic ionic liquid and at least one hydrogen halide (HX) in an apparatus (V1), wherein the hydrogen halide (HX) is removed in gaseous form in an apparatus (V2) from the isomerization product and is at least partly recycled into apparatus (V1).

Ionic liquids are suitable, inter alia, as catalysts for the isomerization of hydrocarbons. A corresponding use of an ionic liquid is described, for example, in WO 2011/069929, where a specific selection of ionic liquids is used in the presence of an olefin for isomerization of saturated hydrocarbons, more particularly for isomerization of methylcyclopentane (MCP) to cyclohexane. A similar process is described in WO 2011/069957, but the isomerization therein is not effected in the presence of an olefin, but with a copper(II) compound.

In addition to the ionic liquid, it is also possible to use hydrogen halides as cocatalysts in isomerization processes. Frequently, the hydrogen halides are used in gaseous form. In order to be able to better utilize the cocatalytic effect of the hydrogen halides, a partial pressure of 1-10 bar of hydrogen halide, especially of hydrogen chloride, is generally established over the reaction mixture in which the isomerization is performed. However, a certain portion of the hydrogen halide used is dissolved in the hydrocarbons and consequently discharged from the isomerization reaction. This proportion of hydrogen halide dissolved in the hydrocarbons has to be removed again from the hydrocarbons after the isomerization, and this removal is in practice frequently associated with problems.

US-A 2011/0155632 discloses a process for preparing products with a low hydrogen halide content, wherein the content of hydrogen halides is reduced in at least two separation steps, by stripping or distillation from a mixture which originates from a reactor and comprises an ionic liquid as a catalyst. In one embodiment of the process described in US-A 2011/0155632, the ionic liquid used as a catalyst is recycled into an alkylation reactor from a downstream phase separator, and hydrogen chloride is recycled from a first distillation column downstream of the phase separator and an isobutane-comprising stream from a second distillation column downstream of the first distillation column into the alkylation reactor. US-A 2011/0155632, however, does not disclose anywhere that a hydrogen halide, especially hydrogen chloride, can be recycled into an isomerization. In addition, in the execution variants described therein, the use of two separation steps, especially of two distillation steps, is absolutely necessary in order to obtain a low content of hydrogen halide in the reaction product. A similar disclosure to that in US-A 2011/0155632 is present in US-A 2011/0155640, but the process described therein relates to a hydrocarbon conversion.

U.S. Pat. No. 3,271,467 discloses a process and a corresponding apparatus for maintaining the hydrogen halide concentration in a hydrocarbon conversion, wherein the catalyst used is a metal halide and the hydrogen halide is used as a promoter. Suitable metal halides are, for example, aluminum chloride, aluminum bromide, boron trifluoride or halides of zinc, tin, antimony or zirconium, but such compounds are not ionic liquids. The hydrocarbon conversion may, for example, be an isomerization of methylcyclopentane (MCP) to cyclohexane. In a (first) stripping apparatus, a stream rich in gaseous hydrogen halide is removed from the hydrocarbon-containing output from the hydrocarbon conversion and discharged from the arrangement. A second stream enriched in hydrogen halide is passed from the stripping apparatus into an absorption apparatus, in order to selectively remove the hydrogen halide present in this stream over a solid absorber therein. The hydrogen halide thus removed is removed again from the solid absorber and recycled into the system.

WO 2010/075038 discloses a process for reducing the content of organic halides in a reaction product which is formed as a result of a hydrocarbon conversion process in the presence of a halogen-comprising catalyst based on an acidic ionic liquid. The hydrocarbon conversion process is especially an alkylation; this process can optionally also be performed as an isomerization. The organic halides are removed from the reaction product by washing with an aqueous alkaline solution. The use of hydrogen halide as a cocatalyst of ionic liquids in isomerization processes and the associated removal of hydrogen halide from the isomerization product, however, is not disclosed in WO 2010/075038.

It is an object of the present invention to provide a novel process for isomerizing at least one hydrocarbon in the presence of an acidic ionic liquid.

The object is achieved by a process for isomerizing at least one hydrocarbon, comprising the following steps:
a) isomerizing at least one hydrocarbon in the presence of an acidic ionic liquid having the composition K1AlₙX₍₃ₙ₊₁₎ where K1 is a monovalent cation, X is halogen and 1<n<2.5, and at least one hydrogen halide (HX) in an apparatus (V1),
b) discharging a mixture (G1) from apparatus (V1), mixture (G1) comprising at least one hydrocarbon and at least one hydrogen halide (HX),
c) feeding mixture (G1) into an apparatus (V2), and drawing off a mixture (G1b) comprising at least 50%, preferably at least 70%, of the hydrogen halide (HX) present in G1 in gaseous form from (V2),
d) partly or fully recycling the mixture (G1b) drawn off in step c) into apparatus (V1).

By virtue of the process according to the invention, it is advantageously possible to remove hydrogen halide present/dissolved in the isomerization product (hydrocarbons) after an isomerization from the isomerization product again. Due to the at least partial recycling of the hydrogen halide, this can be reused in the process.

A further advantage of the process according to the invention is considered to be that, in step c), only a single apparatus (separation stage) is required in order to be able to effectively remove hydrogen halide from the isomerization product, whereas the process according to US-A 2011/0155632 or US-A 2011/0155640 in principle requires two such separation stages, especially distillations. In order to be able to effectively remove any residual amounts of hydrogen halide in the course of the process according to the invention, it is sufficient in the present process to perform a one-stage or optionally multistage wash step with an aqueous medium, for example in the form of a mixer-settler apparatus. Such a step, however, is less complex in apparatus terms and is thus less expensive than the obligatory performance of a second distillation.

### BRIEF DESCRIPTION OF THE FIGURES

FIG. 1 illustrates a preferred embodiment according to the process according to the invention.

FIG. 2 illustrates a further preferred embodiment of the present invention.

FIG. 3 illustrates example 1 schematically.

FIG. 4 illustrates comparative example 2 schematically.

If the apparatus (V2) used in the process according to the invention is a flash apparatus, this is associated with further advantages. The use of a flash apparatus in step c) is first of all less costly and simpler in apparatus terms, especially compared to the use of a rectifying column (due to the corrosiveness of the hydrogen halide, which is particularly disadvantageous given the complex geometries which exist in a column).

The separation effect in the flash apparatus is advantageously achieved merely by lowering the pressure relative to the pressure selected in the isomerization in step a). Thus, no separate energy input is needed, and the corrosiveness of the hydrogen halide is additionally less apparent.

The above-described advantages of the process according to the invention become even more apparent if step c) of the invention is preceded by step g), i.e. upstream connection of a phase separation unit, especially a phase separator, to the apparatus (V2), especially a flash apparatus.

The process according to the invention for isomerization of at least one hydrocarbon in the presence of an acidic ionic liquid and at least one hydrogen halide (HX) with gaseous removal of the hydrogen halide from the isomerization product and at least partial recycling is defined in detail hereinafter.

In the context of the present invention, in step a), at least one hydrocarbon is isomerized in the presence of an acidic ionic liquid having the composition K1AlₙX₍₃ₙ₊₁₎ where K1 is a monovalent cation, X is halogen and 1<n<2.5, and at least one hydrogen halide (HX) in an apparatus (V1). The acidic ionic liquid is used as the isomerization catalyst, and the hydrogen halide as the cocatalyst.

The acidic ionic liquids having the composition K1AlₙX₍₃ₙ₊₁₎ where K1 is a monovalent cation, X is halogen and 1<n<2.5 usable in the context of the present invention are known to those skilled in the art. Such ionic liquids are disclosed (alongside further ionic liquids), for example, in WO 2011/069929. For example, mixtures of two or more ionic acidic liquids may be used, preference being given to using one acidic ionic liquid.

K1 is preferably an unsubstituted or at least partly alkylated ammonium ion or a heterocyclic (monovalent) cation, especially a pyridinium ion, an imidazolium ion, a pyridazinium ion, a pyrazolium ion, an imidazolinium ion, a thiazolium ion, a triazolium ion, a pyrrolidinium ion, an imidazolidinium ion or a phosphonium ion. X is preferably chlorine or bromine.

The acidic ionic liquid more preferably comprises, as a cation, an at least partly alkylated ammonium ion or a heterocyclic cation and/or, as an anion, a chloroaluminate ion having the composition AlₙCl₍₃ₙ₊₁₎ where 1<n<2.5. The at least partly alkylated ammonium ion preferably comprises one, two or three alkyl radicals (each) having 1 to 10 carbon atoms. If two or three alkyl substituents are present with the corresponding ammonium ions, the respective chain length can be selected independently; preferably, all alkyl substituents have the same chain length. Particular preference is given to trialkylated ammonium ions having a chain length of 1 to 3 carbon atoms. The heterocyclic cation is preferably an imidazolium ion or a pyridinium ion.

The acidic ionic liquid especially preferably comprises, as a cation, an at least partly alkylated ammonium ion and, as an anion, a chloroaluminate ion having the composition AlₙCl₍₃ₙ₊₁₎ where 1<n<2.5. Examples of such particularly preferred acidic ionic liquids are trimethylammonium chloroaluminate and triethylammonium chloroaluminate.

The hydrogen halides (HX) used may in principle be any conceivable hydrogen halides, for example hydrogen fluoride (HF), hydrogen chloride (HCl), hydrogen bromide (HBr) or hydrogen iodide (HI). The hydrogen halides can optionally also be used as a mixture, but preference is given in the context of the present invention to using only one hydrogen halide. Preference is given to using the hydrogen halide whose halide moiety is also present in the above-described acidic ionic liquid (at least partly) in the corresponding anion. The hydrogen halide (HX) is preferably hydrogen chloride (HCl) or hydrogen bromide (HBr). The hydrogen halide (HX) is more preferably hydrogen chloride (NCl).

In principle, it is possible in the context of the present invention to use any hydrocarbons, provided that at least one of the hydrocarbons used can be isomerized in the presence of the above-described acidic ionic liquids. On the basis of his or her specialist knowledge, the person skilled in the art knows which hydrocarbons are isomerizable by means of acidic ionic liquids. For example, it is possible to use mixtures of two or more hydrocarbons, but it is also possible to use only one hydrocarbon. Thus, it is possible in the context of the present invention that, in a mixture comprising two or more hydrocarbons, only one of these hydrocarbons is isomerized. Optionally, such mixtures may also comprise compounds which are not themselves hydrocarbons but are miscible therewith.

The hydrocarbon isomerized in step a) is preferably methylcyclopentane (MCP) or a mixture of methylcyclopentane (MCP) with at least one further hydrocarbon selected from cyclohexane, n-hexane, isohexanes, n-heptane, isoheptanes, methylcyclohexane or dimethylcyclopentanes.

More preferably, a mixture of methylcyclopentane (MCP) with at least one further hydrocarbon selected from cyclohexane, n-hexane, isohexanes, n-heptane, isoheptanes, methylcyclohexane or dimethylcyclopentanes is isomerized, the proportion of branched hydrocarbons in the mixture being greater than 50% by weight (based on the sum of all hydrocarbons). Particular preference is given in the context of the present invention to isomerizing methylcyclopentane (MCP) to cyclohexane. The isomerization product obtained in step a) in the process according to the invention is characterized in detail once again hereinafter for step b).

Apparatus (V1) used for performance of the isomerization may in principle be any apparatuses known to those skilled in the art for such a purpose. Apparatus (V1) is preferably a stirred tank or a stirred tank cascade. A “stirred tank cascade” means that two or more, for example three or four, stirred tanks are connected in succession (in series).

The performance of an isomerization of hydrocarbons in the presence of an ionic liquid as a catalyst and a hydrogen halide as a cocatalyst is known to those skilled in the art. The hydrocarbons and the ionic liquid in the isomerization preferably each form a separate phase, though portions of the ionic liquid may be present in the hydrocarbon phase and portions of the hydrocarbons in the ionic liquid phase. The hydrogen halide, especially hydrogen chloride, is introduced, preferably in gaseous form, into the apparatus (V1) for performance of the isomerization. The hydrogen halide may be present, at least in portions, in the two aforementioned liquid phases; the hydrogen halide preferably forms a separate, gaseous phase.

The isomerization is preferably performed at a temperature between 0° C. and 100° C., especially preferably at a temperature between 30° C. and 60° C. It is additionally preferred that the pressure in the isomerization is between 1 and 20 bar abs. (absolute), preferably between 2 and 10 bar abs.

The isomerization is preferably performed in the apparatus (V1) in such a way that two liquid phases and one gaseous phase are present in a stirred tank or a stirred tank cascade. The first liquid phase comprises the acidic ionic liquid to an extent of at least 90% by weight and the second liquid phase comprises the hydrocarbons to an extent of at least 90% by weight. The gas phase comprises at least one hydrogen halide, preferably hydrogen chloride, to an extent of at least 90% by weight. Optionally, a solid phase may also be present, this comprising components from which the ionic liquid is formed in solid form, for example AlCl₃. The pressure and composition of the gas phase are set here such that the partial pressure of the gaseous hydrogen halide, especially of HCl gas, in the gas phase is between 1 and 20 bar abs., preferably between 2 and 10 bar abs.

In step b) of the process according to the invention, a mixture (G1) is discharged from apparatus (V1), mixture (G1) comprising at least one hydrocarbon and at least one hydrogen halide (HX). In other words, this means that mixture (G1) comprises the isomerization product (hydrocarbons) obtained in step a) of the invention in full or at least in part, preferably in full. Mixture (G1) thus differs in terms of (chemical) composition and/or amount of the hydrocarbons present therein from the corresponding hydrocarbon composition present prior to the isomerization. Since the isomerization to be performed in such isomerization processes frequently does not proceed to an extent of 100% (i.e. to completion), the isomerization product generally still also comprises the hydrocarbon to be isomerized (in a smaller amount than before the isomerization). If, for example, MCP is to be isomerized to cyclohexane, the isomerization product frequently comprises a mixture of cyclohexane and (in a smaller amount than before the isomerization) MCP.

As well as the hydrocarbons, mixture (G1) comprises at least one hydrogen halide (HX) and optionally further components. The hydrogen halide (HX) present in mixture (G1) is generally the same hydrogen halide as that used in the above-described isomerization step a). As a further component, mixture (G1) preferably comprises the ionic liquid used in the above-described isomerization step a). Mixture (G1) additionally comprises between 10 and 99% by weight, preferably between 50 and 95% by weight, of acidic ionic liquid (the stated amounts are based on the total weight of hydrocarbons and hydrogen halide in mixture (G1)).

The hydrocarbon present in mixture (G1)—i.e. the isomerization product from step a)—is preferably cyclohexane or a mixture comprising cyclohexane. The hydrocarbon present in mixture (G1) is more preferably a mixture of cyclohexane with at least one further hydrocarbon selected from methylcyclopentane (MCP), n-hexane, isohexanes, n-heptane, isoheptanes, methylcyclohexane and dimethylcyclopentanes.

The hydrocarbon present in mixture (G1) is especially preferably a mixture of cyclohexane, MCP and at least one further hydrocarbon. The further hydrocarbon is preferably selected from n-hexane, isohexanes, n-heptane, isoheptanes, methylcyclohexane and dimethylcyclopentanes. More particularly, the proportion of branched hydrocarbons in mixture (G1) (i.e. after the isomerization) is less than 10% by weight (based on the sum of all hydrocarbons present in mixture (G1)).

In a preferred embodiment of the present invention, mixture (G1) comprises i) as a hydrocarbon a mixture of cyclohexane with at least one further hydrocarbon selected from methylcyclopentane (MCP), n-hexane, isohexanes, n-heptane, isoheptanes, methylcyclohexane and dimethylcyclopentanes, ii) hydrogen chloride (HCl) and iii) an acidic ionic liquid which has, as a cation, an at least partly alkylated ammonium ion and, as an anion, a chloroaluminate ion having the composition AlₙCl₍₃ₙ₊₁₎ where 1<n<2.5.

In step c) of the process according to the invention, mixture (G1) is fed into an apparatus (V2), and a mixture (G1b) comprising at least 50% of the hydrogen halide (HX) present in G1 is drawn off in gaseous form from (V2), Mixture (G1b) preferably comprises at least 70%, more preferably at least 95%, especially preferably at least 99%, of hydrogen halide (based in each case on the corresponding amount in G1).

The apparatus (V2) used to perform the gaseous drawing-off (removal) of the hydrogen halide (HX) from mixture (G1) may in principle be any apparatus known for such a purpose to the person skilled in the art, preferably a concentration apparatus, rectifying column, an apparatus for flash vaporization (flash apparatus) or a stripping apparatus. Apparatus (V2) is intended, in the context of the process according to the invention, preferably to perform removal of the hydrogen halides from the hydrocarbons. V2 is especially preferably a flash apparatus or a stripping apparatus.

In the context of the present invention, step c) should be understood such that, in the case of use of a flash apparatus as apparatus (V2), an appropriate flash operation (flashing) is performed with mixture (G1). The same applies to the further configurations of apparatus (V2) detailed below, such as stripping apparatus or vaporizer.

In the context of the present invention, the term “concentration”, which is performed in a corresponding concentration apparatus, is understood to mean the following: a characteristic feature of concentration is that a portion of the liquid mixture to be separated is vaporized with supply of heat and is condensed after removal from the remaining liquid mixture. For the original liquid phase, a vapor phase is thus produced, in which the relatively low-boiling mixture components are enriched.

In the context of the present invention, the term “rectification”, which is performed in a corresponding rectifying column (rectifying apparatus), also called rectification column or rectification apparatus, is understood to mean the following: in rectification, the vapor produced by distillation is conducted in countercurrent to a portion of the condensate thereof in a rectifying column. In this way, less volatile components are enriched in the top product and more volatile components in the bottom product of the rectifying column.

In the context of the present invention, the term “flashing”, which is performed in a corresponding flash apparatus and can also be referred to as flash vaporization, is understood to mean the following: Flash vaporization (flashing) involves decompressing a liquid mixture into a suitable apparatus (flash apparatus), for example into a vapor/liquid separation vessel (i.e., in a suitable apparatus, for example a valve, a lowering of the pressure finds off, this being sufficient to cause a portion of the liquid mixture to vaporize spontaneously). The liquid mixture may originate, for example, from a reaction stage operated at higher pressure. However, it is also possible to effect preheating in a preheater, for example to boiling temperature, in which case the pressure in the preheater must be higher than the pressure in the downstream separation vessel. The vapor forming in the course of decompression has a higher proportion of relatively low-boiling components than the mixture entering the separator. The flash evaporation thus ensures partial separation of the incoming mixture, in which case the separator can act as a sole theoretical plate. The flashing can also be combined with heat supply to the liquid mixture which remains in the flashing operation, for example by means of a circulation vaporizer connected to the separation vessel.

In the context of the present invention, the term “stripping”, which is performed in a corresponding stripping apparatus, is understood to mean the following: in the course of stripping, one or more relatively low-boiling components are depleted from a liquid, these being contacted, preferably in a countercurrent column, with gases such as nitrogen, air or steam, such that the decrease in the partial pressure of the relatively low-boiling components in the gas phase brought about by the gas results in a decrease in the solubility thereof in the liquid.

Further information regarding the above terms “distillation”, “rectification”, “vaporization”, “flashing” and/or “stripping” can be found in the following textbooks: Sattler, Thermische Trennverfahren [Thermal Separation Processes], VCH, 1988; Perry's Chemical Engineers' Handbook, 7th edition; R. H. Perry, D. W. Green, 1997, McGraw-Hill.

In step d) of the process according to the invention, the mixture (G1b) drawn off in step c) is partly or fully recycled into apparatus (V1), optionally with a pressure increase by means of a suitable apparatus, for example a jet compressor, piston compressor, turbo compressor or screw compressor. The mixture (G1b) drawn off in step c) is preferably recycled fully into apparatus (V1). If complete recycling of mixture (G1b) is not performed, the excess amounts of mixture (G1b) are discharged from the process according to the invention and (generally) discarded.

In the process according to the invention, step c) is preferably performed in such a way that a one-stage vaporization, preferably a flash vaporization, takes place in apparatus (V2).

In a preferred embodiment of the present invention, in addition to the above-described steps a) to d), the following additional steps e) and f) are conducted, these being defined as follows:
e) discharging a mixture (G2) from apparatus (V2), mixture (G2) comprising at least one hydrocarbon and an amount of at least one hydrogen halide (HX) reduced relative to mixture (G1) by at least 50%,
f) washing mixture (G2) with an aqueous medium to obtain a mixture (G3) comprising at least one hydrocarbon and not more than 100 ppm by weight, preferably not more than 10 ppm by weight, of hydrogen halide (HX) (based on the total weight of (G3)).

Preferably (according to step e)), mixture (G2) comprises an amount of at least one hydrogen halide (HX) reduced relative to mixture (G1) by at least 70%, more preferably at least 95%, especially preferably at least 99%.

In the context of the process according to the invention, it is preferable that mixture (G3) obtained in process step f) (wash step f), with regard to the composition and/or amount of the hydrocarbons present therein, corresponds completely or at least substantially to mixtures (G1) and (G2). The expression “corresponds substantially” shall be understood in this context to mean that at least 90% by weight, preferably at least 95% by weight, especially at least 99% by weight, of the amount of hydrocarbons present in mixture (G1) is also present in mixture (G3). Especially preferably, mixture (G3) does not comprise any further components apart from at least one hydrocarbon and not more than 100 ppm by weight, preferably not more than 10 ppm by weight of hydrogen halide.

Preference is given to performing the additional process step f) in such a way that the wash according to step f) comprises at least two wash steps:
f1) in a first wash step, the aqueous medium used has a pH>9, preferably>12,
f2) in a second wash step, the aqueous medium used has a pH between 5 and 9, preferably between 6 and 8.

The aqueous medium in the first wash step preferably comprises an alkali metal hydroxide, especially preferably NaOH. The aqueous medium in the second wash step is preferably water, especially preferably demineralized water.

Optionally, additional process step f) can be performed in such a way that step f2) can be performed prior to step f1). In this process variant, washing is thus effected first with an aqueous medium of relatively low pH, followed by into the wash with an aqueous medium of higher pH. In addition, it is also possible to perform several steps f1) and several steps f2) in succession, optionally in alternating sequence.

Preference is given in the context of the present invention to performing wash step f) in two stages, first step f1) and then step f2).

In one embodiment of the present invention, only a one-stage wash step f) is performed, in which case the aqueous medium has a pH of 5 to 9, preferably between 6 and 8, and is especially preferably demineralized water. In this embodiment, wash step f) is preferably performed in an extraction column operated in countercurrent or a mixer-settler arrangement.

In addition, it is preferable in the context of the present invention that step f) is performed using at least one dispersion and phase separation unit or at least one extraction column per wash stage. The dispersion and phase separation unit is preferably a mixer-settler apparatus (combination of a stirred tank with a downstream phase separator), a combination of static mixers with phase separators or a combination of mixing pump with phase separator.

It is additionally preferred in the context of the present invention that, in the case of a multistage, especially two-stage, wash, mixture (G2) is conducted in countercurrent to the aqueous medium. It is especially preferred in the context of the present invention that mixture (G2) discharged from the apparatus (V2) is washed with the aqueous medium (according to step f)) without any intermediate steps.

In another embodiment, wash step f) is performed in a multistage mixer-settler apparatus, preferably operated in countercurrent, or extraction is effected with water in an extraction column operated in countercurrent. In the case of the mixer-settler apparatus or extraction column, a further wash stage is preferably connected downstream thereof in flow direction of mixture (G2) (comprising the hydrocarbons), this being fed with fresh water. In the aqueous outlet thereof is an apparatus for continuous measurement of the pH or the electrical conductivity, in order thus to monitor the complete removal of the non-hydrocarbon components, especially HCl.

FIG. 1 once again illustrates the process according to the invention in a preferred embodiment. In this embodiment, the two process steps e) and f) are also performed. Apparatus (V1) is preferably a stirred tank or a stirred tank cascade; apparatus (V2) is preferably a vaporizer, especially a flash apparatus. The arrow coming from apparatus (V2) and pointing upward shows that it is optionally also possible in the context of the process according to the invention, in step d), to perform only partial recycling of the mixture (G1b) removed in step c), or that mixture (G1b) is supplied to a further process step, preferably to a material separation. This is especially advantageous when (V2) is performed as a stripping operation. In this case, full or partial separation of the hydrogen halide from the stripping gas used can be performed in the process step, and the hydrogen halide-enriched stream thus obtained can be recycled fully or partly into the apparatus (V1). According to FIG. 1, the mixture (G2) discharged from the apparatus (V2) is washed with the aqueous medium (according to step f)) without any intermediate steps. In FIG. 1, wash step f) of the invention is referred to in simplified form with the abbreviation “W”. Wash step f) according to FIG. 1 may, as described above, be performed in one or more stages, in which case preference is given to performing a multistage, especially two-stage, wash of mixture (G2) in countercurrent to the aqueous medium, and/or a dispersion and phase separation unit, especially a mixer-settler apparatus, is used.

In a further preferred embodiment of the present invention, a further step g) is performed between step b) and step c), this being defined as follows:
g) feeding mixture (G1) into a phase separation unit, especially into a phase separator, and removing at least 90%, preferably at least 99%, of the acidic ionic liquid from mixture (G1) in the phase separation unit, and feeding the mixture (G1) depleted of the acidic ionic liquid into apparatus (V2).

It is additionally preferred that the acidic ionic liquid removed from mixture (G1) in the phase separation unit (according to step g)) is recycled partly or fully into apparatus (V1).

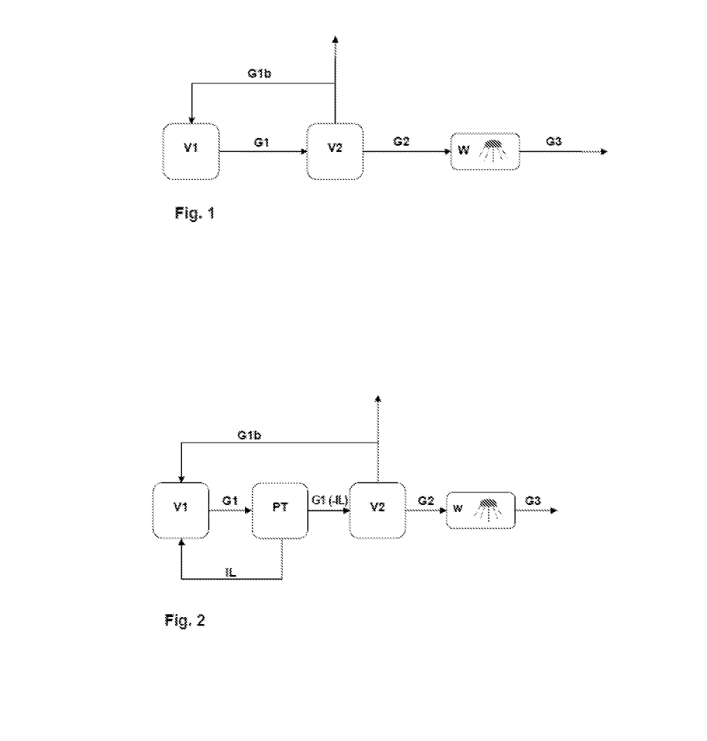

The above-described further preferred embodiment of the present invention is additionally illustrated in FIG. 2. In FIG. 2, the abbreviations, arrows and other symbols have similar meanings to those explained above for FIG. 1; PT means phase separation unit, IL means acidic ionic liquid.

In the context of the present invention, cyclohexane is preferably isolated from mixture (G3). Processes and apparatuses for removal of cyclohexane from mixture (G3) are known to those skilled in the art.

In a further preferred embodiment of the present invention, apparatus (V1) is a stirred tank or a stirred tank cascade and is operated at a pressure (p1) of 1 to 10 bar. It is additionally preferred that apparatus (V2) is a flash apparatus and is operated at a pressure (p2) less than the pressure (p1) in apparatus (V1). Due to the use of the flash apparatus, the pressure is lowered relative to the pressure present in the isomerization according to step a). The pressure values (p2) can be selected arbitrarily (for example 1 to 5 bar), provided that they are lower than the pressure (p1) present in apparatus (V1) in the course of isomerization.

The present invention is to be illustrated hereinafter by examples.

For the simulation calculation, BASF's own software Chemasin was used (in the case of use of the commercially available software Aspen Plus (manufacturer: AspenTech, Burlington/Mass., USA), the same results would be obtained). The following substances or mixtures are used for the example calculation:

a) hydrocarbon mixture (A) having the composition

| | |
|---|---|
| 2-methylpentane | 0.66% by wt. |
| 3-methylpentane | 1.95% by wt. |
| n-hexane | 28.85% by wt. |
| methylcyclopentane | 51.02% by wt. |
| cyclohexane | 17.44% by wt. |
| 2,3-dimethylbutane | 0.05% by wt. |
| other hydrocarbons | 0.03% by wt. |

b) chloride gas (B)

c) liquid (IL), specifically trimethylammonium chloroaluminate (TMA-IL)

For the examples described, (A) and (B) are mixed in such a way that the resulting mixture after the phase separation G1(-IL) has an HCl content of 1.5% by weight.

### 1. Example with HCl Removal or Recycling Via Flash Apparatus (V2)

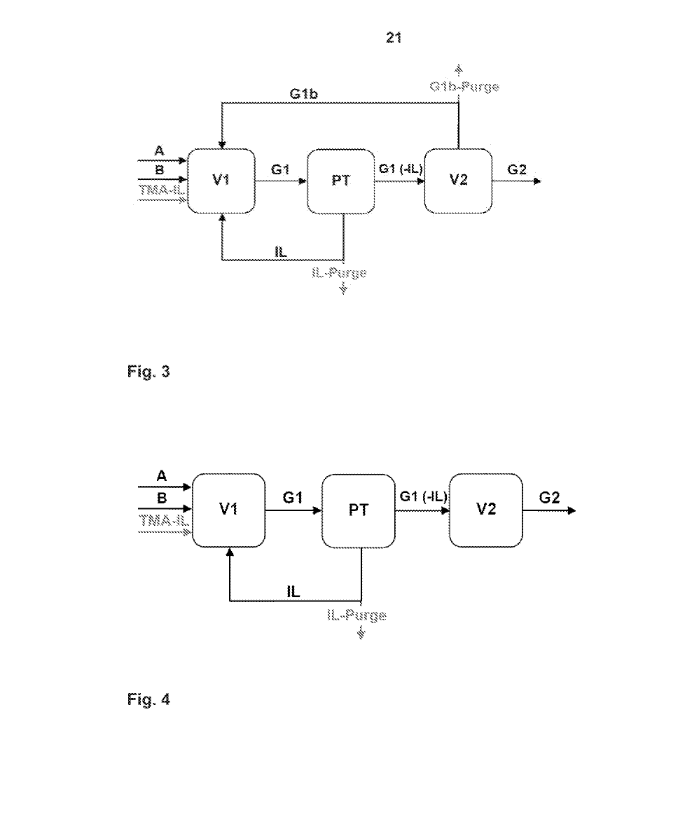

Example 1 is shown schematically in FIG. 3. In an apparatus V1, an isomerization of a hydrocarbon mixture (A) takes place in the presence of an ionic liquid (trimethylammonium chloroaluminate—TMA-IL) which serves as a catalyst. The volume ratio of ionic liquid to organic phase is 5 l/l. In addition, hydrogen chloride gas (B) for stabilization of the IL and the recycle streams G1b from a flash apparatus V2 and IL from a phase separation are fed in. For apparatus V1, an operating pressure of 3.5 bar (abs) and a temperature of 50° C. are assumed. The resulting mixture G1 is passed into an apparatus for phase separation PT. In a simplification, it is assumed for this apparatus that the TMA-IL is removed and recycled completely and as a pure IL phase. The organic phase G1(-IL) is passed into the flash apparatus V2, where it is decompressed to an operating pressure of 1 bar (abs). The resulting gas fraction G1b is recycled into the apparatus V1. The “purge” refers to the substreams of G1 and IL which can optionally be removed from the process. The liquid mixture G2 is discharged from V2 and can be treated in a subsequent step by washing (not part of this example calculation).

The calculated properties and compositions of the streams are shown in table 1.

TABLE 1
Properties and composition of the streams from example 1

| Stream: | | | A | B | G1 | IL | G1(-IL) | G1b | G2 |
|---|---|---|---|---|---|---|---|---|---|
| from apparatus | | | | | V1 | PT | PT | V2 | V2 |
| to apparatus | | | V1 | V1 | PT | V1 | V2 | V1 | |
| Phase | | | fluid | gas | fluid | fluid | fluid | gas | fluid |
| | | | cur. | cur. | cur. | cur. | cur. | cur. | cur. |
| Properties | | Unit | value | value | value | value | value | value | value |
| Temperature | | ° C. | 45.000 | 50.000 | 50.000 | 50.000 | 50.000 | 45.404 | 45.404 |
| Pressure | | bar | 3.500 | 3.500 | 3.500 | 3.500 | 3.500 | 1.000 | 1.000 |
| Enthalpy | | kW | 0.138 | 0.002 | 1.511 | 1.369 | 0.142 | 0.017 | 0.124 |
| Mean molar mass | | kg/kmol | 84.791 | 36.461 | 274.964 | 362.251 | 83.141 | 55.089 | 84.408 |
| Thermal conductivity | | W/(m*K) | 0.113 | 0.015 | 0.143 | 0.150 | 0.112 | 0.014 | 0.114 |
| Viscosity eta | | mPa*s | 0.349 | 0.016 | 6.067 | 21.012 | 0.396 | 0.011 | 0.442 |
| Surface tension | | N/m | 0.018 | | 0.014 | 0.013 | 0.018 | | 0.019 |
| Specific heat | | kJ/kg/K | 2.114 | 0.799 | 2.009 | 2.000 | 2.095 | 1.220 | 2.073 |
| Density | | kg/m³ | 695.521 | 4.750 | 1268.900 | 1385.511 | 702.646 | 2.080 | 709.586 |
| Mass flow rate | | kg/h | 4.983 | 0.017 | 54.435 | 49.287 | 5.147 | 0.147 | 5.000 |
| | | | cur. | cur. | cur. | cur. | cur. | cur. | cur. |
| Concentrations | MolM. | Unit | value | value | value | value | value | value | value |
| 2-M-PENTANE | 86.179 | g/g | 0.0066 | | 0.0043 | | 0.0457 | 0.0429 | 0.0458 |
| 3-M-PENTANE | 86.179 | g/g | 0.0195 | | 0.0039 | | 0.0408 | 0.0348 | 0.0410 |
| HEXANE | 86.179 | g/g | 0.2885 | | 0.0197 | | 0.2084 | 0.1538 | 0.2100 |
| M-CY-PENTANE | 84.163 | g/g | 0.5102 | | 0.0114 | | 0.1210 | 0.0765 | 0.1223 |
| CYCLOHEXANE | 84.163 | g/g | 0.1744 | | 0.0521 | | 0.5514 | 0.2646 | 0.5599 |
| 2,2-DM-BUTANE | 86.179 | g/g | 0.0000 | | 0.0003 | | 0.0037 | 0.0048 | 0.0036 |
| 2,3-DM-BUTANE | 86.179 | g/g | 0.0005 | | 0.0013 | | 0.0137 | 0.0147 | 0.0137 |
| TMA-IL | 362.251 | g/g | | | 0.9054 | 1.0000 | | | |
| HCL | 36.461 | g/g | | 1.0000 | 0.0014 | | 0.0150 | 0.4079 | 0.0034 |

### 2. Comparative Example Without HCl Removal or Recycling

Comparative example 2 is shown schematically in FIG. 4. In an apparatus V1, an isomerization of a hydrocarbon mixture (A) takes place in the presence of an ionic liquid (trimethylammonium chloroaluminate—TMA-IL), which serves as a catalyst. The volume ratio of ionic liquid to organic phase is 5 l/l. In addition, hydrogen chloride gas (B) for stabilization of the IL is fed in. For apparatus V1, an operating pressure of 3.5 bar (abs) and a temperature of 50° C. are assumed. The resulting mixture G1 is passed into an apparatus for phase separation PT. In a simplification, it is assumed for this apparatus that the TMA-IL is removed and recycled completely and as a pure IL phase. The organic phase G1(-IL) is passed into an apparatus V2 which has no function compared to example 1. As a result, the lack of influence of a flash apparatus is to be illustrated. The liquid mixture G2, which in this case corresponds to the composition of mixture G1(-IL), is discharged from V2 and can be treated by washing in a subsequent step.

The calculated properties and compositions of the streams are shown in table 2.

TABLE 2
Properties and composition of the streams from comparative example 2

| Stream: | | | A | B | G1 | IL | G1(-IL) | G2 |
|---|---|---|---|---|---|---|---|---|
| from apparatus | | | | | V1 | PT | PT | V2 |
| to apparatus | | | V1 | V1 | PT | V1 | V2 | |
| Phase | | | fluid | gas | fluid | fluid | fluid | fluid |
| | | | cur. | cur. | cur. | cur. | cur. | cur. |
| Properties | | Unit | value | value | value | value | value | value |
| Temperature | | ° C. | 50.000 | 50.000 | 50.000 | 50.000 | 50.000 | 50.000 |
| Pressure | | bar | 3.500 | 3.500 | 3.500 | 3.500 | 3.500 | 1.000 |
| Enthalpy | | kW | 0.137 | 0.008 | 1.500 | 1.363 | 0.138 | 0.138 |
| Mean molar mass | | kg/kmol | 84.791 | 36.461 | 276.413 | 362.251 | 83.138 | 83.138 |
| Thermal conductivity | | W/(m*K) | 0.113 | 0.015 | 0.143 | 0.150 | 0.113 | 0.113 |
| Viscosity eta | | mPa*s | 0.349 | 0.016 | 6.196 | 21.012 | 0.396 | 0.396 |
| Surface tension | | N/m | 0.018 | | 0.014 | 0.013 | 0.018 | 0.018 |
| Specific heat | | kJ/kg/K | 2.114 | 0.799 | 2.009 | 2.000 | 2.094 | 2.094 |
| Density | | kg/m³ | 695.526 | 4.750 | 1271.289 | 1385.511 | 702.834 | 702.834 |
| Mass flow rate | | kg/h | 4.925 | 0.075 | 54.054 | 49.054 | 5.000 | 5.000 |
| | | | cur. | cur. | cur. | cur. | cur. | cur. |
| Concentrations | MolM. | Unit | value | value | value | value | value | value |
| 2-M-PENTANE | 86.179 | g/g | 0.0066 | | 0.0042 | | 0.0450 | 0.0450 |
| 3-M-PENTANE | 86.179 | g/g | 0.0195 | | 0.0037 | | 0.0404 | 0.0404 |
| HEXANE | 86.179 | g/g | 0.2889 | | 0.0193 | | 0.2083 | 0.2083 |
| M-CY-PENTANE | 84.163 | g/g | 0.5101 | | 0.0112 | | 0.1212 | 0.1212 |
| CYCLOHEXANE | 84.163 | g/g | 0.1742 | | 0.0511 | | 0.5528 | 0.5528 |
| 2,2-DM-BUTANE | 86.179 | g/g | 0.0000 | | 0.0003 | | 0.0036 | 0.0036 |
| 2,3-DM-BUTANE | 86.179 | g/g | 0.0005 | | 0.0012 | | 0.0135 | 0.0135 |
| TMA-IL | 362.251 | g/g | | | 0.9075 | 1.0000 | | |
| HCL | 36.461 | g/g | | 1.0000 | 0.0014 | | 0.0150 | 0.0150 |

### 3. Results of the Simulation Calculation

The results show that, through the feeding of the mixture G1(-IL) into an apparatus V2 (especially into a flash apparatus) under the defined simulation conditions, 77.9% of the hydrogen chloride present in G1(-IL) is drawn off and fully recycled. The subsequent treatment of the mixture G2, which removes residual amounts of hydrogen halide by means of washing, is made easier as a result. Use of a flash apparatus reduces the HCl content in G2 from 1.50% by weight to 0.34% by weight, which results in a potential saving in the amount of washing composition used.

In addition, the hydrogen halide, owing to the recycling, can be reused in the process to an extent of more than 75%. In the case of full recycling of gas stream G1b, there are additionally no accompanying losses of product of value.

The use of a flash apparatus is less expensive and simpler in apparatus terms compared to distillative processes, and is less prone to corrosion because of less complex geometries, and, moreover, no separate energy input is necessary.

1-20. (canceled)

21. A process for isomerizing at least one hydrocarbon, comprising the following steps:
a) isomerizing at least one hydrocarbon in the presence of an acidic ionic liquid having the composition K1AlₙX₍₃ₙ₊₁₎ where K1 is a monovalent cation, X is halogen and 1<n<2.5, and at least one hydrogen halide (HX) in an apparatus (V1),
b) discharging a mixture (G1) from apparatus (V1), mixture (G1) comprising at least one hydrocarbon and at least one hydrogen halide (HX),
c) feeding mixture (G1) into an apparatus (V2), and drawing off a mixture (G1b) comprising at least 50% of the hydrogen halide (HX) present in G1 in gaseous form from (V2),
d) partly or fully recycling the mixture (G1b) drawn off in step c) into apparatus (V1).

22. A process according to claim 21, wherein in step c) the mixture (G1b) comprises at least 70% of the hydrogen halide (HX) present in G1.

23. The process according to claim 21, wherein the apparatus (V2) is a concentration apparatus, a rectifying column, a flash apparatus or a stripping apparatus.

24. The process according to claim 23, wherein the apparatus (V2) is a flash apparatus or a stripping apparatus.

25. The process according to claim 21, wherein the hydrogen halide (HX) is hydrogen chloride (HCl).

26. The process according to claim 21, wherein methylcyclopentane (MCP) is isomerized to cyclohexane.

27. The process according to claim 21, further comprising steps e) and f):
e) discharging a mixture (G2) from apparatus (V2), mixture (G2) comprising at least one hydrocarbon and an amount of at least one hydrogen halide (HX) reduced relative to mixture (G1) by at least 50%, and
f) washing mixture (G2) with an aqueous medium to obtain a mixture (G3) comprising at least one hydrocarbon and not more than 100 ppm by weight of hydrogen halide (HX) (based on the total weight of (G3)).

28. The process according to claim 27, wherein in step e) the amount of at least one hydrogen halide (HX) is reduced relative to mixture (G1) by at least 99% or in step f) the mixture (G3) comprises not more than 10 ppm by weight of hydrogen halide (HX).

29. The process according to claim 27, wherein the wash according to step f) comprises at least two wash steps:
f1) in a first wash step, the aqueous medium used has a pH>9,
f2) in a second wash step, the aqueous medium used has a pH between 5 and 9, and optionally to perform step f2) prior to step f1).

30. The process according to claim 29, wherein the aqueous medium in step f1) comprises NaOH or the aqueous medium in step f2) is demineralized water.

31. The process according to claim 27, wherein the aqueous medium has a pH of 5 to 9.

32. The process according to claim 27, wherein step f) is performed using at least one dispersion and phase separation unit or at least one extraction column per wash stage.

33. The process according to claim 32, wherein the dispersion and phase separation unit is a mixer-settler apparatus, a combination of static mixers with phase separators or a combination of mixing pump with phase separator.

34. The process according to claim 32, wherein, in a multistage wash, mixture (G2) is conducted in countercurrent to the aqueous medium.

35. The process according to claim 21, wherein a one-stage vaporization takes place in apparatus (V2).

36. The process according to claim 35, wherein the vaporization is a flash vaporization.

37. The process according to claim 27, wherein mixture (G2) discharged from apparatus (V2) is washed with the aqueous medium without any intermediate steps.

38. The process according to claim 21, wherein mixture (G1) comprises, as the hydrocarbon, cyclohexane or a mixture of cyclohexane with at least one further hydrocarbon selected from methylcyclopentane (MCP), n-hexane, isohexanes, n-heptane, isoheptanes, methylcyclohexane or dimethylcyclopentanes.

39. The process according to claim 21, wherein the acidic ionic liquid comprises, as a cation, an at least partly alkylated ammonium ion or a heterocyclic cation or, as an anion, a chloroaluminate ion having the composition AlₙCl₍₃ₙ₊₁₎ where 1<n<2.5.

40. The process according to claim 21, wherein mixture (G1) additionally comprises between 10 and 99% by weight.

41. The process according to claim 21, wherein a further step g) is performed between step b) and step c):
g) feeding mixture (G1) into a phase separation unit, and removing at least 90% of the acidic ionic liquid from mixture (G1) in the phase separation unit, and feeding the mixture (G1) depleted of the acidic ionic liquid into apparatus (V2).

42. The process according to claim 41, wherein the acidic ionic liquid removed from mixture (G1) in the phase separation unit is recycled partly or fully into apparatus (V1).

43. The process according to claim 27, wherein cyclohexane is isolated from mixture (G3).

44. The process according to claim 21, wherein apparatus (V1) is a stirred tank or a stirred tank cascade and is operated at a pressure (p1) of 1 to 10 bar.

45. The process according to claim 21, wherein apparatus (V2) is a flash apparatus and is operated at a pressure (p2) less than the pressure (p1) in apparatus (V1).
